SEQUENCE LISTING (3 sequences)

SEQ ID NO: 1 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 2 - DNA, 1624 bp, Homo sapiens
gttgcttggatcagtctaggtgcagctgccggatccttcagcgtctgcatctcggcgtcgccccgcgtaccgtcgcccggctctccgccgctctcccggggtttcggggcacttgggtcccacagtctggtcctgcttcaccttcccctgacctgagtagtcgccatggcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgactaacgtgtcacggggaagagctccctgctgtgtgcctgcaccaccccactgccttccttcagcacctttagctgcatttgtatgccagtgcttaacacattgccttattcatactagcatgctcatgaccaacacatacacgtcatagaagaaaatagtggtgcttctttctgatctctagtggagatctctttgactgctgtagtactaaagtgtacttaatgttactaagtttaatgcctggccattttccatttatatatattttttaagaggctagagtgcttttagccttttttaaaaactccatttatattacatttgtaaccatgatactttaatcagaagcttagccttgaaattgtgaactcttggaaatgttattagtgaagttcgcaactaaactaaacctgtaaaattatgatgattgtattcaaaagattaatgaaaaataaacatttctgtccccctgaaaaaaaaaaaaaaaaaaaaaaaaaaa

SEQ ID NO: 3 - DNA, 963 bp, Homo sapiens
atggcgcaagttttacgtgggacggttaccgattttccgggctttgatgaacgcgcggatgctgagaccttacgcaaagcgatgaagggcctgggtactgacgaggagtccattctgaccctcctgacctcgcgctctaacgctcaacgccaagaaattagtgcagcctttaagactctgttcggacgtgatttgctggacgatcttaaatcagaattaactggcaagtttgaaaaactgattgttgcactcatgaaaccgtcgcgcctgtatgatgcatatgaactgaaacacgctctgaagggcgcgggtactaatgagaaagtgctgactgaaatcattgcgtcccgtaccccagaagaactgcgcgccattaagcaagtttatgaggaggagtacggttctagcctggaagatgatgtcgtaggggatacaagtggttattatcaacgtatgctggttgtgctcctgcaggcgaaccgcgacccagatgcgggcattgacgaagcccaagtcgaacaagatgcgcaggctctgttccaagcaggcgagctgaaatggggtaccgacgaagaaaaattcattacgatctttggcacacgtagcgttagccacttacggaaagttttcgataagtacatgacgatcagcggatttcagattgaagaaaccatcgaccgtgagacgtctggtaatctggagcagttactgctggcagttgttaaaagcattcgctccattccggcgtatttggctgagacgctgtattatgcaatgaagggtgccggcaccgatgatcacacccttattcgcgtgatggtgtctcgttcggaaattgatctgtttaacattcggaaagagtttcgtaagaatttcgcgacgtcattatattcgatgattaaaggtgacacttccggcgattacaagaaagcgttgctgttattgtgcggtgaagatgattaa

(19) United States
(12) Patent Application Publication
(10) Pub. No.: US 2023/0374091 A1
(43) Pub. Date: Nov. 23, 2023

(54) ANNEXIN A5 COMPOSITIONS AND METHODS

(71) Applicant: London Health Sciences Centre Research Inc., London (CA)

(72) Inventors: Qingping Feng, London (CA); Xiangru Lu, London (CA); Peter B. Stathopulos, London (CA); Stephen Barr, London (CA)

(21) Appl. No.: 18/031,063

(22) PCT Filed: Oct. 8, 2021
(86) PCT No.: PCT/CA21/51422

§ 371 Date: Apr. 10, 2023

Related U.S. Application Data
(60) Provisional application No. 63/089,584, filed on Oct. 9, 2020.

(51) Int. Cl. C07K 14/47; A61P 31/14

(52) U.S. Cl. CPC C07K 14/4721; A61P 31/14; A61K 38/00

(57) ABSTRACT

Described herein in aspects is annexin A5 for interacting with angiotensin converting enzyme 2 (ACE2) and/or for preventing and/or treating a coronavirus infection. Also described is a method for preventing and/or treating an ACE2-mediated infection and/or a coronavirus infection, the method comprising administering annexin A5 to a subject in need thereof.

### FIELD

The present invention relates to ACE2. In particular, the present invention relates to ACE2 and annexin A5 interactions as well as related compositions and methods.

### BACKGROUND

Coronavirus disease 2019 (COVID-19) is a respiratory tract infection caused by a novel coronavirus, SARS-CoV-2. In severe cases, the disease is complicated by acute respiratory distress syndrome (ARDS), sepsis and septic shock, and multi-organ failure, including the lungs and heart. Studies have shown that endothelial injury, sepsis and thromboinflammatory response are key contributors to the development of ARDS and multi-organ failure in COVID-19 patients.^{6,7} Currently, there is no proven therapy to effectively prevent or treat COVID-19. Remdesivir was shown to shorten time to recovery without a significant survival benefit,^{1,2}while hydroxychloroquine was associated with longer time to clinical recovery and less overall clinical recovery.³

Angiotensin converting enzyme 2 (ACE2) is an enzyme attached to the cell membranes of cells located in the lungs, arteries, heart, kidney, and intestines. ACE2 serves as the entry point into cells for some coronaviruses, such as SARS-CoV-2. ACE2 is also known to lower blood pressure by catalyzing the hydrolysis of angiotensin II (a vasoconstrictor peptide) into angiotensin (a vasodilator).

Annexin A5 (or annexin V) is a cellular protein in the annexin group. In flow cytometry, annexin V is commonly used to detect apoptotic cells by its ability to bind to phosphatidylserine, a marker of apoptosis when it is on the outer leaflet of the plasma membrane. Annexin A5 (A5) is an endogenous protein with potent anti-inflammatory and anti-apoptotic and anticoagulant/antithrombotic properties. In addition, A5 also binds to heparan sulfate, a major component of endothelial glycocalyx, and thereby inhibits endothelial production of thrombin. Recombinant human A5 inhibits proinflammatory responses and improves animal survival in rodent models of bacterial sepsis. U.S. Pat. No. 9,192,649 describes compositions comprising an effective amount of Annexin A5 for use in treatment of an inflammatory disorder and for use in improving organ function. Methods for administering such compositions for treatment of animals are also provided.

### SUMMARY OF THE INVENTION

In accordance with an aspect, there is provided annexin A5 for interacting with angiotensin converting enzyme 2 (ACE2).

In an aspect, the annexin A5 binds to ACE2.

In an aspect, annexin A5 binding to ACE2 does not substantially affect ACE2 enzyme activity.

In an aspect, the annexin A5 inhibits interaction of another molecule with ACE2, such as a virus or viral protein.

In an aspect, the annexin A5 inhibits interaction of a coronavirus or coronavirus protein with ACE2.

In an aspect, the annexin A5 inhibits interaction of a coronavirus spike protein with ACE2.

In accordance with an aspect, there is provided annexin A5 as a SARS-CoV-2 research tool.

In accordance with an aspect, there is provided annexin A5 as an ACE2 research tool.

In an aspect, the annexin A5 is human.

In an aspect, the annexin A5 is recombinant.

In an aspect, the annexin A5 comprises an amino acid sequence having at least 80% sequence identity to SEQ ID NO:1:

MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD

or an active fragment thereof.

In an aspect, the annexin A5 comprises the amino acid sequence of SEQ ID NO:1.

In an aspect, the annexin A5 consists of the amino acid sequence of SEQ ID NO:1.

In an aspect, the annexin A5 is bound to a moiety, such as a therapeutic agent, detectable agent, and/or targeting agent, optionally wherein the moiety is a peptide or small molecule.

In accordance with an aspect, there is provided a composition comprising the annexin A5 described herein and a pharmaceutically acceptable carrier.

In an aspect, the composition is an inhalable composition or a nasal spray.

In accordance with an aspect, there is provided a method for preventing and/or treating SARS-CoV-2, the method comprising administering annexin A5 to a subject in need thereof.

In an aspect, the annexin A5 is human.

In an aspect, the annexin A5 is recombinant.

In an aspect, the annexin A5 comprises an amino acid sequence having at least 80% sequence identity to SEQ ID NO:1:

MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD

or an active fragment thereof.

In an aspect, the annexin A5 comprises the amino acid sequence of SEQ ID NO:1.

In an aspect, the annexin A5 consists of the amino acid sequence of SEQ ID NO:1.

In an aspect, the annexin A5 is bound to a moiety, such as a therapeutic agent, detectable agent, and/or targeting agent, optionally wherein the moiety is a peptide or small molecule.

In an aspect, the annexin A5 is inhaled or a nasal spray.

In an aspect, the subject has a chronic condition such as hypertension or diabetes, is over age 60, and/or is taking an ACE inhibitor.

In an aspect, the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.01 to about 1 μg/ml, once, twice, three, or 4 times or more daily or once, twice, three, four, five, or six times weekly, or upon appearance of symptoms, or upon a suspected or known exposure.

In accordance with an aspect, there is provided a method for preventing and/or treating a coronavirus infection and/or for reducing viral load in a coronavirus infection, the method comprising administering annexin A5 to a subject in need thereof.

In an aspect, the coronavirus infection comprises HCoV-229E, HCoV-0043, HCoV-NL63, HCoV-HKU1, SARS-CoV, MERS-CoV, or SARS-CoV-2.

In an aspect, the annexin A5 is human.

In an aspect, the annexin A5 is recombinant.

In an aspect, the annexin A5 comprises an amino acid sequence having at least 80% sequence identity to SEQ ID NO:1:

MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD

or an active fragment thereof.

In an aspect, the annexin A5 comprises the amino acid sequence of SEQ ID NO:1.

In an aspect, the annexin A5 consists of the amino acid sequence of SEQ ID NO:1.

In an aspect, the annexin A5 is bound to a moiety, such as a therapeutic agent, detectable agent, and/or targeting agent, optionally wherein the moiety is a peptide or small molecule.

In an aspect, the annexin A5 is inhaled or a nasal spray.

In an aspect, the subject has a chronic condition such as hypertension or diabetes, is over age 60, and/or is taking an ACE inhibitor.

In an aspect, the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.01 to about 1 μg/ml, once, twice, three, or 4 times or more daily or once, twice, three, four, five, or six times weekly, or upon appearance of symptoms, or upon a suspected or known exposure.

In accordance with an aspect, there is provided a method for preventing and/or treating a symptom of a coronavirus infection, the method comprising administering annexin A5 to a subject in need thereof.

In an aspect, the coronavirus infection comprises HCoV-229E, HCoV-0C43, HCoV-NL63, HCoV-HKU1, SARS-CoV, MERS-CoV, or SARS-CoV-2.

In an aspect, the annexin A5 is human.

In an aspect, the annexin A5 is recombinant.

In an aspect, the annexin A5 comprises an amino acid sequence having at least 80% sequence identity to SEQ ID NO:1:

MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD

or an active fragment thereof.

In an aspect, the annexin A5 comprises the amino acid sequence of SEQ ID NO:1.

In an aspect, the annexin A5 consists of the amino acid sequence of SEQ ID NO:1.

In an aspect, the annexin A5 is bound to a moiety, such as a therapeutic agent, detectable agent, and/or targeting agent, optionally wherein the moiety is a peptide or small molecule.

In an aspect, the annexin A5 is inhaled or a nasal spray.

In an aspect, the symptom comprises sepsis, septic shock, acute respiratory distress syndrome (ARDS), and/or organ failure, such as heart and/or lung failure.

In an aspect, the subject has a chronic condition such as hypertension or diabetes, is over age 60, and/or is taking an ACE inhibitor.

In an aspect, the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.01 to about 1 μg/ml, once, twice, three, or 4 times or more daily or once, twice, three, four, five, or six times weekly, or upon appearance of symptoms, or upon a suspected or known exposure.

In accordance with an aspect, there is provided a method for binding to angiotensin converting enzyme 2 (ACE2), the method comprising administering annexin A5 in vitro or in vivo.

In an aspect, the annexin A5 is human.

In an aspect, the annexin A5 is recombinant.

In an aspect, the annexin A5 comprises an amino acid sequence having at least 80% sequence identity to SEQ ID NO:1:

MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD

or an active fragment thereof.

In an aspect, the annexin A5 comprises the amino acid sequence of SEQ ID NO:1.

In an aspect, the annexin A5 consists of the amino acid sequence of SEQ ID NO:1.

In an aspect, the annexin A5 is bound to a moiety, such as a therapeutic agent, detectable agent, and/or targeting agent, optionally wherein the moiety is a peptide or small molecule.

In an aspect, the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.01 to about 1 μg/ml, once, twice, three, or 4 times or more daily or once, twice, three, four, five, or six times weekly, or upon appearance of symptoms, or upon a suspected or known exposure.

In accordance with an aspect, there is provided a method for preventing and/or treating an ACE2-mediated infection, the method comprising administering annexin A5 to a subject in need thereof.

In an aspect, the ACE2-mediated infection is a coronavirus.

In an aspect, the coronavirus is HCoV-229E, HCoV-0C43, HCoV-NL63, HCoV-HKU1, SARS-CoV, MERS-CoV, or SARS-CoV-2.

In an aspect, the annexin A5 is human.

In an aspect, the annexin A5 is recombinant.

In an aspect, the annexin A5 comprises an amino acid sequence having at least 80% sequence identity to SEQ ID NO:1, or an active fragment thereof.

In an aspect, the annexin A5 comprises the amino acid sequence of SEQ ID NO:1.

In an aspect, the annexin A5 consists of the amino acid sequence of SEQ ID NO:1.

In an aspect, the annexin A5 is bound to a moiety, such as a therapeutic agent, detectable agent, and/or targeting agent, optionally wherein the moiety is a peptide or small molecule.

In an aspect, the annexin A5 is inhaled or a nasal spray.

In an aspect, the subject has a chronic condition such as hypertension or diabetes, is over age 60, and/or is taking an ACE inhibitor.

In an aspect, the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.01 to about 1 μg/ml, once, twice, three, or 4 times or more daily or once, twice, three, four, five, or six times weekly, or upon appearance of symptoms, or upon a suspected or known exposure.

The novel features of the present invention will become apparent to those of skill in the art upon examination of the following detailed description of the invention. It should be understood, however, that the detailed description of the invention and the specific examples presented, while indicating certain aspects of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

The present invention will be further understood from the following description with reference to the Figure, in which:

FIG. 1. (A) The ACE2 extracellular domain (blue cartoon ribbons) forms a concave cleft in three-dimensional space. (B) The residues that putatively bind A5 create a contiguous region inside the ACE2 cleft (magenta spheres). (C) Zoomed view identifying the 20 residues forming the contiguous A5 interacting region. (D) A5 (green cartoon ribbons) helices are arranged perpendicular to the long axis of the structure. (E) The surface that putatively interacts with ACE2 (magenta spheres) is localized to one face of A5. This face is located on the same side as the 4 calcium (Ca²⁺) binding sites of A5, suggesting that Ca²⁺ could regulate the interaction. (F) Twenty-six residues on A5 are predicted to mediate interactions with ACE2. (G) Crystal structure of the ACE2 extracellular domain (blue cartoon ribbons) in complex with the C-terminal domain of the SARS-COV2 spike protein (orange cartoon ribbon).

FIG. 2. Inhibition of spike protein receptor binding domain (RBD) to angiotensin converting enzyme 2 (ACE2) binding by recombinant human annexin A5. Data are mean±SEM of 3 independent tests using ACE2: spike RBD (SARS-CoV-2) inhibitor screening assay kit (Catalog #79936, BPS Bioscience, San Diego, CA). Annexin A5 at 2.5 μg/ml inhibited spike-RBD to ACE2 binding by 46%.

FIG. 3. Representative images of human 293T-ACE2 cells infected with or without SARS-CoV-2 in the presence of recombinant human annexin A5 (A5). Cells were infected with SARS-CoV-2 at an MOI of 0.01 for 2 hours in the absence of A5. After 2 hours, virus was removed, and cells were incubated with fresh medium containing A5 for 72 hours. Brightfield images were obtained after 72 hours of infection using an EVOS M7000 imaging system (Thermo Fisher Scientific). Six independent and adjacent images were taken at the center of each well and stitched together using ImageJ (v2.1.0/1.53c).

FIG. 4. Effects of recombinant human annexin A5 (A5) on survival of human 293T-ACE2 cells infected with SARS-CoV-2. Cells were infected with SARS-CoV-2 at an MOI of 0.01 for 2 hours in the absence of A5. After 2 hours, virus was removed, and cells were incubated with fresh medium containing 2-fold dilutions of A5 for 72 hours. Percent survival was measured using Cell Titer Glo (Promega).

FIG. 5. Solution nuclear magnetic resonance (NMR) spectroscopic analysis of the effect of annexin A5 on the spike protein:ACE2 interaction. A. Coomassie-blue stained SDS-PAGE gel showing successful human ACE2 and spike-RBD protein expression for use in this study. B. Size exclusion chromatography of ¹⁵N-spike-RBD, highlighting the exceptional yield and non-aggregated and primarily monodisperse nature of the sample. C. Overlaid ¹H-¹⁵N-HSQC spectra showing profound differences in amide (¹H-¹⁵N) chemical environments of ¹⁵N-spike-RBD in isolation (blue), in complex with ACE2 (magenta) and mixed with ACE2+Anx5 (green). Direct ACE2 binding is indicated by changes in ¹H-¹⁵N peak intensities and positions (black arrows). A structural effect of annexin A5 on the ¹⁵N-spike-RBD:ACE2 interaction is indicated by complex-specific ¹H-¹⁵N peak perturbations (hollow arrows).

FIG. 6. Effects of human recombinant annexin A5 on the SARS-CoV-2 nucleocapsid (N) gene expression. Human 293T-ACE2 cells were infected with WT (A) or B.1.1.7 variant (B) SARS-CoV-2 at an MOI of 0.01 or 0.001, respectively in the presence of annexin A5 (0-5 μg/ml) for 48 hours. N gene mRNA levels were analyzed by qPCR from 2 independent experiments (WT: IC50=0.036 μg/ml; B.1.1.7 variant: IC50=0.010 μg/ml). The data indicate annexin A5 effectively lowers the viral load in both WT and B.1.1.7 variant infected cells.

### DETAILED DESCRIPTION

### Definitions

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Although any methods and materials similar or equivalent to those described herein can be used in the practice for testing of the present invention, the typical materials and methods are described herein. In describing and claiming the present invention, the following terminology will be used.

It is also to be understood that the terminology used herein is for the purpose of describing particular aspects only and is not intended to be limiting. Many patent applications, patents, and publications may be referred to herein to assist in understanding the aspects described. Each of these references is incorporated herein by reference in its entirety.

In understanding the scope of the present application, the articles “a”, “an”, “the”, and “said” are intended to mean that there are one or more of the elements. Additionally, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

It will be understood that any aspects described as “comprising” certain components may also “consist of” or “consist essentially of,” wherein “consisting of” has a closed-ended or restrictive meaning and “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Typically, a composition consisting essentially of a set of components will comprise less than 5% by weight or volume, typically less than 3% by weight, more typically less than 1%, and even more typically less than 0.1% by weight of non-specified component(s).

It will be understood that any component defined herein as being included may be explicitly excluded from the claimed invention by way of proviso or negative limitation.

In addition, all ranges given herein include the end of the ranges and also any intermediate range points, whether explicitly stated or not.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

Although methods and materials similar or equivalent to those described herein can be used in the practice or testing of this disclosure, suitable methods and materials are described below. The abbreviation, “e.g.” is derived from the Latin exempli gratia and is used herein to indicate a non-limiting example. Thus, the abbreviation “e.g.” is synonymous with the term “for example.” The word “or” is intended to include “and” unless the context clearly indicates otherwise.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Variants” are biologically active constructs, polypeptides, or fragments thereof having an amino acid sequence that differs from a comparator sequence by virtue of an insertion, deletion, modification and/or substitution of one or more amino acid residues within the comparative sequence. Variants generally have less than 100% sequence identity with the comparative sequence. Ordinarily, however, a biologically active variant will have an amino acid sequence with at least about 70% amino acid sequence identity with the comparative sequence, such as at least about 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity. The variants include peptide fragments of at least 10 amino acids that retain some level of the biological activity of the comparator sequence. Variants also include polypeptides wherein one or more amino acid residues are added at the N- or C-terminus of, or within, the comparative sequence. Variants also include polypeptides where a number of amino acid residues are deleted and optionally substituted by one or more amino acid residues. Variants also may be covalently modified, for example by substitution with a moiety other than a naturally occurring amino acid or by modifying an amino acid residue to produce a non-naturally occurring amino acid.

“Percent amino acid sequence identity” is defined herein as the percentage of amino acid residues in the candidate sequence that are identical with the residues in the sequence of interest, such as the polypeptides of the invention, after aligning the sequences and introducing gaps, if necessary, to achieve the maximum percent sequence identity, and not considering any conservative substitutions as part of the sequence identity. None of N-terminal, C-terminal, or internal extensions, deletions or insertions into the candidate sequence shall be construed as affecting sequence identity or homology. Methods and computer programs for the alignment are well known in the art, such as “BLAST”.

The constructs described herein may include modifications. Such modifications include, but are not limited to, conjugation to an effector molecule. Modifications further include, but are not limited to conjugation to detectable reporter moieties. Modifications that extend half-life (e.g., pegylation) are also included. Proteins and non-protein agents may be conjugated to the constructs by methods that are known in the art. Conjugation methods include direct linkage, linkage via covalently attached linkers, and specific binding pair members (e.g., avidin-biotin). Such methods include, for example, that described by Greenfield et al., Cancer Research 50, 6600-6607 (1990), which is incorporated by reference herein and those described by Amon et al., Adv. Exp. Med. Biol. 303, 79-90 (1991) and by Kiseleva et al, Mol. Biol. (USSR)25, 508-514 (1991), both of which are incorporated by reference herein.

“Active” or “activity” for the purposes herein refers to a biological activity of the annexin A5 described herein, wherein “biological” activity refers to a biological function (either inhibitory or stimulatory) caused by the annexin A5, for example, binding to ACE2 and/or inhibiting binding of a pathogen to ACE2.

The terms “therapeutically effective amount”, “effective amount” or “sufficient amount” mean a quantity sufficient, when administered to a subject, including a mammal, for example a human, to achieve a desired result, for example an amount effective to treat and/or prevent an infection or the symptoms thereof. Effective amounts of the compounds described herein may vary according to factors such as the pathogen, or the age, sex, and weight of the subject. Dosage or treatment regimes may be adjusted to provide the optimum therapeutic response, as is understood by a skilled person. For example, administration of a therapeutically effective amount of the annexin A5 described herein is, in aspects, sufficient to treat and/or prevent an infection, such as SARS-COV-2, and/or the symptoms thereof.

Moreover, a treatment regime of a subject with a therapeutically effective amount may consist of a single administration, or alternatively comprise a series of applications. The length of the treatment period depends on a variety of factors, such as the pathogen, the age of the subject, the concentration of the agent, the responsiveness of the patient to the agent, or a combination thereof. It will also be appreciated that the effective dosage of the agent used for the treatment may increase or decrease over the course of a particular treatment regime. Changes in dosage may result and become apparent by standard diagnostic assays known in the art. The annexin A5 described herein may, in aspects, be administered before, during or after treatment with conventional therapies for the disease or disorder in question, such as an antiviral agent.

The term “subject” as used herein refers to any member of the animal kingdom, typically a mammal. The term “mammal” refers to any animal classified as a mammal, including humans, other higher primates, domestic and farm animals, and zoo, sports, or pet animals, such as dogs, cats, cattle, horses, sheep, pigs, goats, rabbits, etc. Typically, the mammal is human.

Administration “in combination with” one or more further therapeutic agents includes simultaneous (concurrent) and consecutive administration in any order.

The term “pharmaceutically acceptable” means that the compound or combination of compounds is compatible with the remaining ingredients of a formulation for pharmaceutical use, and that it is generally safe for administering to humans according to established governmental standards, including those promulgated by the United States Food and Drug Administration.

The term “pharmaceutically acceptable” carrier includes, but is not limited to solvents, dispersion media, coatings, antibacterial agents, antifungal agents, isotonic and/or absorption delaying agents and the like. The use of pharmaceutically acceptable carriers is well known.

### Compositions and Methods

Described herein is an in silico analysis that shows a putative interaction between annexin A5 and human angiotensin converting enzyme 2 (ACE2), a receptor of SARS-CoV-2 and other pathogens for host cell entry. The predicted interaction between A5 and ACE2 may cause allosteric inhibition to SARS-CoV-2 binding to ACE2. Due to this novel mechanism of action of annexin A5, annexin A5 has now been identified as a prophylactic agent that can be used to protect against infection by such pathogens and also as a novel treatment modality.

Annexin A5 is well-suited for use as a treatment for COVID-19 because it has multifaceted actions which include binding to ACE2 and inhibition of proinflammatory response, coagulation, and thrombosis. Thus, annexin A5 can prevent SARS-CoV-2 entry into cells and therefore prevent and treat the infection directly and also target some of the harmful symptoms associated with COVID-19, such as acute respiratory distress syndrome (ARDS), sepsis and septic shock, and multi-organ failure, including the lungs and heart.

Thus, described herein is annexin A5 for interacting with angiotensin converting enzyme 2 (ACE2). It will be understood that annexin A5 can be used in vitro or in vivo for this purpose and that annexin A5 can be used as a research tool for studying coronaviruses, such as SARS-CoV-2 and/or for studying ACE2.

The annexin A5 described herein may interact directly with ACE2, through direct binding to ACE2. In aspects, this binding may have little to no effect on ACE2 enzyme activity. In additional or alternative aspects, the annexin A5 may interact indirectly with ACE2, through its interaction with another molecule that itself interacts with ACE2. For example, some viruses or viral proteins are known to use ACE2 to enter cells. Annexin A5 may interact with ACE2 directly to reduce or prevent virus entry or it may interact with the virus itself to reduce or prevent virus entry. In typical aspects, the annexin A5 inhibits interaction of a coronavirus or coronavirus protein with ACE2. In more typical aspects, the annexin A5 inhibits interaction of a coronavirus spike protein with ACE2.

The annexin A5 described herein may be of any species, however, typically human annexin A5 is used when the subject is human. The annexin A5 is typically recombinant and comprises an amino acid sequence having at least about 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO:1:

MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASR
TPEELRAIKQVYEEEYGSSLEDDVVGDTSGYYQRMLVVLL
QANRDPDAGIDEAQVEQDAQALFQAGELKWGTDEEKFITI
FGTRSVSHLRKVFDKYMTISGFQIEETIDRETSGNLEQLL
LAVVKSIRSIPAYLAETLYYAMKGAGTDDHTLIRVMVSRS
EIDLFNIRKEFRKNFATSLYSMIKGDTSGDYKKALLLLCG
EDD

or an active fragment thereof.

In aspects, the annexin A5 is provided alone and in other aspects, the annexin A5 is bound, such as covalently linked, to another moiety. For example, the annexin A5 may be provided as a fusion protein, where the annexin A5 amino acid sequence is bound to another sequence. In aspects, the other moiety is, for example, a therapeutic agent, a detectable agent, and/or a targeting agent. The other moiety may be any type of moiety that can be bound to annexin A5, for example, it may be a peptide or a small molecule.

Also provided herein are compositions, such as therapeutic compositions, comprising annexin A5 and a pharmaceutically acceptable carrier or excipient. The composition may be configured for any form of use. In typical aspects, however, the composition is an inhalable composition or a nasal spray.

Methods and uses for annexin A5 are also described herein. For example, provided herein is a method for preventing and/or treating SARS-CoV-2. The method comprises administering annexin A5 as described herein to a subject in need thereof. In aspects, the subject may be afflicted with a chronic condition, such as hypertension or diabetes. In additional or alternative aspects, the subject may be over about age 60. In additional or alternative aspects, the subject may be taking an ACE inhibitor.

Also provided herein is a method for preventing and/or treating a coronavirus infection. Further provided is a method for reducing viral load in a coronavirus infection. Also provided is a method for preventing and/or treating a symptom of a coronavirus infection, which may be associated with or independent from the prevention and/or treatment of a coronavirus infection. These methods comprise administering annexin A5 to a subject in need thereof.

Also provided herein are methods for binding to angiotensin converting enzyme 2 (ACE2). The method comprises administering annexin A5 in vitro or in vivo. It will be understood that the methods for binding to ACE2 may be for the purpose of treating and/or preventing a disease or for research or diagnostic purposes. In other aspects, provided herein are methods for preventing and/or treating an ACE2-mediated infection. This method comprises administering annexin A5 to a subject in need thereof.

The ACE2-mediated infection described herein may be any infection in which ACE2 is used by the infectious agent to enter cells and/or cause symptoms of disease. An example of an ACE2-mediated infection is a coronavirus infection. For example, the coronavirus infection may be HCoV-229E, HCoV-OC43, HCoV-NL63, HCoV-HKU1, SARS-CoV, MERS-CoV, and/or SARS-CoV-2. Variants of any such coronaviruses are also encompassed herein, such as the following:
Alpha (B.1.1.7 and Q lineages)
Beta (B.1.351 and descendent lineages)
Delta (B.1.617.2 and AY lineages)
Gamma (P.1 and descendent lineages)
Epsilon (B.1.427 and B.1.429)
Eta (B.1.525)
Iota (B.1.526)
Kappa (B.1.617.1)
1.617.3
Mu (B.1.621, B.1.621.1)
Zeta (P.2)

In methods whereby the annexin A5 is used to treat and/or prevent a coronavirus symptom, the symptom may be, for example, sepsis, septic shock, acute respiratory distress syndrome (ARDS), and/or organ failure, such as heart and/or lung failure. Also contemplated are coagulation, thrombosis, and an inflammatory response.

In certain aspects, the subject being administered annexin A5 may be of any age and may have co-morbidities or be otherwise healthy. In some aspects, the subject is a pediatric subject or an adult subject. In some aspects, the subject is an elderly subject, such as aged 60 or older, such as age 65, 70, or 75 or older. The subject in aspects may have a chronic condition, such as hypertension or diabetes and in aspects may be taking medications to treat another condition. In some aspects, the subject is taking an ACE inhibitor.

It will be understood that the annexin A5 may be administered at any suitable dose, depending upon the desired use and condition to treated/prevented. In aspects, the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml. For example, from about 0.1 to about 1 μg/ml. Similarly, the frequency of dosing can be titrated as desired depending on the subject, use, and condition. For example, the dosing can be once, twice, three, or 4 times or more daily or once, twice, three, four, five, or six times weekly. In aspects, the annexin A5 may be administered upon appearance of symptoms of an ACE2-mediated infection or a coronavirus infection and/or upon a suspected or known exposure to such an infection.

Sequences that are substantially identical to the above sequences are also contemplated, such as those that are at least about 80, 81, 82, 83, 84, 85, 86, 87, 88, 89, 90, 91, 92, 93, 94, 95, 96, 97, 98, or 99% identical. Fragments of the sequences or the substantially identical variant sequences are also contemplated herein.

A substantially identical sequence may comprise one or more conservative amino acid mutations. It is known in the art that one or more conservative amino acid mutations to a reference sequence may yield a mutant peptide with no substantial change in physiological, chemical, or functional properties compared to the reference sequence; in such a case, the reference and mutant sequences would be considered “substantially identical” polypeptides. Conservative amino acid mutation may include addition, deletion, or substitution of an amino acid; a conservative amino acid substitution is defined herein as the substitution of an amino acid residue for another amino acid residue with similar chemical properties (e.g. size, charge, or polarity).

In a non-limiting example, a conservative mutation may be an amino acid substitution. Such a conservative amino acid substitution may substitute a basic, neutral, hydrophobic, or acidic amino acid for another of the same group. By the term “basic amino acid” it is meant hydrophilic amino acids having a side chain pK value of greater than 7, which are typically positively charged at physiological pH. Basic amino acids include histidine (His or H), arginine (Arg or R), and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of Eisenberg (1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G).

“Acidic amino acid” refers to hydrophilic amino acids having a side chain pK value of less than 7, which are typically negatively charged at physiological pH. Acidic amino acids include glutamate (Glu or E), and aspartate (Asp or D).

Sequence identity is used to evaluate the similarity of two sequences; it is determined by calculating the percent of residues that are the same when the two sequences are aligned for maximum correspondence between residue positions. Any known method may be used to calculate sequence identity; for example, computer software is available to calculate sequence identity. Without wishing to be limiting, sequence identity can be calculated by software such as NCBI BLAST2 service maintained by the Swiss Institute of Bioinformatics (and as found at ca.expasy.org/tools/blast/), BLAST-P, Blast-N, or FASTA-N, or any other appropriate software that is known in the art.

The substantially identical sequences of the present invention may be at least 85% identical; in another example, the substantially identical sequences may be at least 70, 75, 80, 85, 90, 95, 96, 97, 98, 99, or 100% (or any percentage there between) identical at the amino acid level to sequences described herein. In specific aspects, the substantially identical sequences retain the activity and specificity of the reference sequence. In a non-limiting embodiment, the difference in sequence identity may be due to conservative amino acid mutation(s).

Also encompassed herein are isolated or purified polypeptides, or fragments thereof immobilized onto a surface using various methodologies; for example, and without wishing to be limiting, the polypeptides may be linked or coupled to the surface via His-tag coupling, biotin binding, covalent binding, adsorption, and the like. The solid surface may be any suitable surface, for example, but not limited to the well surface of a microtiter plate, channels of surface plasmon resonance (SPR) sensorchips, membranes, beads (such as magnetic-based or sepharose-based beads or other chromatography resin), glass, a film, or any other useful surface.

Also described herein are nucleic acid molecules encoding the constructs described herein, as well as vectors comprising the nucleic acid molecules and host cells comprising the vectors.

The annexin A5 described herein can be fused to additional amino acid residues. Such amino acid residues can be a peptide tag to facilitate isolation, for example. Other amino acid residues for homing of the antibodies to specific organs or tissues are also contemplated.

Any suitable method or route can be used to administer the annexin A5 described herein. Routes of administration include, for example, oral, intravenous, intraperitoneal, subcutaneous, or intramuscular administration. In particular, nasal and inhalation routes of administration are contemplated, such as nasal sprays and inhalable compositions to treat and/or prevent a respiratory infection, such as a SARS-CoV-2 infection.

It is understood that the annexin A5 described herein, where used in a mammal for the purpose of prophylaxis or treatment, will typically be administered in the form of a composition additionally comprising a pharmaceutically acceptable carrier. Suitable pharmaceutically acceptable carriers include, for example, one or more of water, saline, phosphate buffered saline, dextrose, glycerol, ethanol and the like, as well as combinations thereof. Pharmaceutically acceptable carriers may further comprise minor amounts of auxiliary substances such as wetting or emulsifying agents, preservatives or buffers, which enhance the shelf life or effectiveness of the annexin A5. The compositions of the injection may, as is well known in the art, be formulated so as to provide quick, sustained or delayed release of the active ingredient after administration to the mammal.

The compositions described herein may be administered over a period of hours, days, weeks, or months, depending on several factors, including the severity and type of the infection or other condition being treated, whether a recurrence is considered likely, or to prevent the infection or other condition, etc. The administration may be constant, e.g., constant infusion over a period of hours, days, weeks, months, etc. Alternatively, the administration may be intermittent, e.g., the compositions may be administered once a day over a period of days, once an hour over a period of hours, or any other such schedule as deemed suitable.

The compositions described herein can be prepared by per se known methods for the preparation of pharmaceutically or cosmetically acceptable compositions which can be administered to subjects, such that an effective quantity of the active substance is combined in a mixture with a pharmaceutically acceptable vehicle. Suitable vehicles are described, for example, in “Handbook of Pharmaceutical Additives” (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456 (the entirety of which is incorporated herein by reference).

Pharmaceutically acceptable carriers are well known to those skilled in the art and include, for example, sterile saline, lactose, sucrose, calcium phosphate, gelatin, dextrin, agar, pectin, peanut oil, olive oil, sesame oil, cannabis oil, and water. Furthermore the composition may comprise one or more stabilizers such as, for example, carbohydrates including sorbitol, mannitol, starch, sucrose, dextrin and glucose, proteins such as albumin or casein, and buffers like alkaline phosphates.

The compositions described herein can, in embodiments, be administered for example, by parenteral, intravenous, subcutaneous, intradermal, intramuscular, intracranial, intraorbital, ophthalmic, intraventricular, intracapsular, intraspinal, intracisternal, intraperitoneal, intranasal, intrarectal, intravaginal, inhalational, nasally, aerosol, oral, topical, or transdermal administration. Typically, the compositions of the invention are administered by inhalation or nasally.

It is understood by one of skill in the art that the compositions described herein can be used in conjunction with known therapies for prevention and/or treatment of an infection, for example, in subjects. The compositions described herein may, in embodiments, be administered in combination, concurrently or sequentially, with conventional treatments for infections and/or their symptoms. The compositions described herein may be formulated together with such conventional treatments when appropriate.

The following examples do not include detailed descriptions of conventional methods, such as those employed in the construction of vectors and plasmids, the insertion of genes encoding polypeptides into such vectors and plasmids, or the introduction of plasmids into host cells. Such methods are well known to those of ordinary skill in the art and are described in numerous publications including Sambrook, J., Fritsch, E. F. and Maniatis, T. (1989), Molecular Cloning: A Laboratory Manual, 2nd edition, Cold Spring Harbor Laboratory Press, which is incorporated by reference herein.

Without further description, it is believed that one of ordinary skill in the art can, using the preceding description and the following illustrative examples, make and utilize the compounds of the present invention and practice the claimed methods. The following working examples therefore, specifically point out the typical aspects of the present invention and are not to be construed as limiting in any way in the remainder of the disclosure.

### EXAMPLES

### Example 1: Putative Interactions Between the Human Angiotensin Converting Enzyme-Related Carboxypeptidase (ACE2) and Human Annexin A5

### Methods

In Silico Analysis: The human ACE2 (1R42.pdb) and Ca²⁺-loaded human annexin V (1ANX.pdb) crystal structure atomic coordinates were used as inputs for the in silico predictions, executed using the default parameters. The human ACE2:SARS-COV2 spike protein complex structure (6LZG.pdb) was used to compare the relative locations of the A5 and spike protein binding sites on ACE2. Structure images were rendered using the 1R42, 1ANX and 6LZG coordinate files in PyMOL (Schrodinger, LLC).

Results:

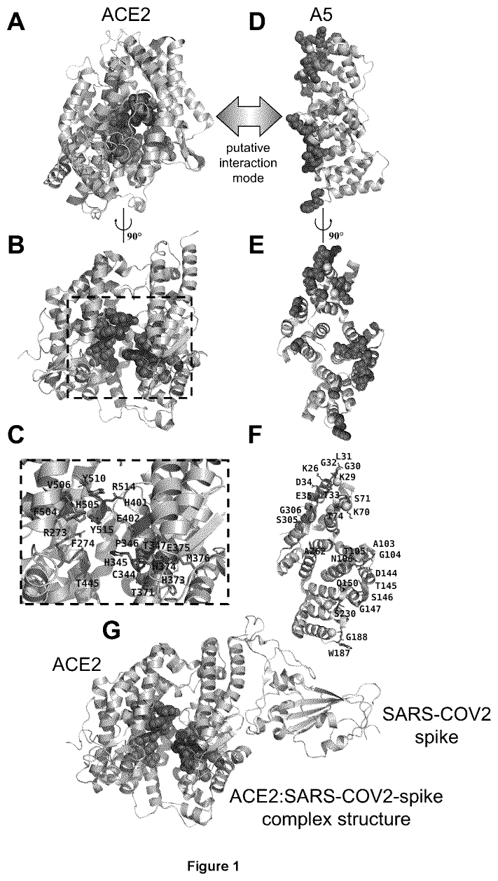

FIG. 1 shows putative interactions between the human angiotensin converting enzyme-related carboxypeptidase (ACE2) and human annexin 5 (A5), suggesting allosteric inhibition of SARS-COV2 spike protein binding. (A) The ACE2 extracellular domain (blue cartoon ribbons) forms a concave cleft in three-dimensional space. (B) The residues that putatively bind A5 create a contiguous region inside the ACE2 cleft (magenta spheres). (C) Zoomed view identifying the 20 residues forming the contiguous A5 interacting region. (D) A5 (green cartoon ribbons) helices are arranged perpendicular to the long axis of the structure. (E) The surface that putatively interacts with ACE2 (magenta spheres) is localized to one face of A5. This face is located on the same side as the 4 calcium (Ca²⁺) binding sites of A5, suggesting that Ca²⁺ could regulate the interaction. (F) Twenty-six residues on A5 are predicted to mediate interactions with ACE2. (G) Crystal structure of the ACE2 extracellular domain (blue cartoon ribbons) in complex with the C-terminal domain of the SARS-COV2 spike protein (orange cartoon ribbon). In (A-F), the ACE2:A5 Interaction sites were identified in silico using the BIPSPI webserver.¹ In (B and C), the dashed boxes encompass similar regions on ACE2. In (D-F), the Ca²⁺ ions are shown as yellow spheres. In (A and D), the molecules are oriented to show the compatible interaction mode via movement along the horizontal plane. In (G), the putative A5 interaction site (magenta spheres) is inside the ACE2 cleft, whereas the SARS-COV2 spike protein interaction site is outside the cleft. The human ACE2 (1R42.pdb)² and Ca²⁺-loaded human annexin V (1ANX.pdb)³ crystal structure atomic coordinates were used as inputs for the in silico predictions, executed using the default parameters. The human ACE2:SARS-COV2 spike protein complex structure (6LZG.pdb)⁴ was used to compare the relative locations of the A5 and spike protein binding sites on ACE2. Structure images were rendered using the 1R42, 1ANX and 6LZG coordinate files in PyMOL (Schrodinger, LLC).

### References

1. Sanchez-Garcia R, Sorzano C O S, Carazo J M, Segura J. BIPSPI: a method for the prediction of partner-specific protein-protein interfaces. Bioinformatics. 2019; 35:470-477.
2. Towler P, Staker B, Prasad S G, Menon S, Tang J, Parsons T, Ryan D, Fisher M, Williams D, Dales N A, Patane M A, Pantoliano M W. ACE2 X-ray structures reveal a large hinge-bending motion important for inhibitor binding and catalysis. J Biol Chem. 2004; 279:17996-18007.
3. Sopkova J, Renouard M, Lewit-Bentley A. The crystal structure of a new high-calcium form of annexin V. J Mol Biol. 1993; 234:816-825.
4. Wang Q, Zhang Y, Wu L, Niu S, Song C, Zhang Z, Lu G, Qiao C, Hu Y, Yuen K-Y, Wang Q, Zhou H, Yan J, Qi J. Structural and Functional Basis of SARS-CoV-2 Entry by Using Human ACE2. Cell. 2020; 181:894-904.e9.

### Example 2: Binding Experiments

### Methods

Human annexin A5 mRNA sequence (Accession #NM_001154):

| | |
|---|---|
| 1 | gttgcttgga tcagtctagg tgcagctgcc |
| | ggatccttca gcgtctgcat ctcggcgtcg |
| 61 | ccccgcgtac cgtcgcccgg ctctccgccg |
| | ctctcccggg gtttcggggc acttgggtcc |
| 121 | cacagtctgg tcctgcttca ccttcccctg |
| | acctgagtag tcgccatggc acaggttctc |
| 181 | agaggcactg tgactgactt ccctggattt |
| | gatgagcggg ctgatgcaga aactcttcgg |
| 241 | aaggctatga aaggcttggg cacagatgag |
| | gagagcatcc tgactctgtt gacatcccga |
| 301 | agtaatgctc agcgccagga aatctctgca |
| | gcttttaaga ctctgtttgg cagggatctt |
| 361 | ctggatgacc tgaaatcaga actaactgga |
| | aaatttgaaa aattaattgt ggctctgatg |
| 421 | aaaccctctc ggctttatga tgcttatgaa |
| | ctgaaacatg ccttgaaggg agctggaaca |
| 481 | aatgaaaaag tactgacaga aattattgct |
| | tcaaggacac ctgaagaact gagagccatc |
| 541 | aaacaagttt atgaagaaga atatggctca |
| | agcctggaag atgacgtggt gggggacact |
| 601 | tcagggtact accagcggat gttggtggtt |
| | ctccttcagg ctaacagaga ccctgatgct |
| 661 | ggaattgatg aagctcaagt tgaacaagat |
| | gctcaggctt tatttcaggc tggagaactt |
| 721 | aaatggggga cagatgaaga aaagtttatc |
| | accatctttg gaacacgaag tgtgtctcat |
| 781 | ttgagaaagg tctttgacaa gtacatgact |
| | atatcaggat ttcaaattga ggaaaccatt |
| 841 | gaccgcgaga cttctggcaa tttagagcaa |
| | ctactccttg ctgttgtgaa atctattcga |
| 901 | agtatacctg cctaccttgc agagaccctc |
| | tattatgcta tgaagggagc tgggacagat |
| 961 | gatcataccc tcatcagagt catggtttcc |
| | aggagtgaga ttgatctgtt taacatcagg |
| 1021 | aaggagttta ggaagaattt tgccacctct |
| | ctttattcca tgattaaggg agatacatct |
| 1081 | ggggactata agaaagctct tctgctgctc |
| | tgtggagaag atgactaacg tgtcacgggg |
| 1141 | aagagctccc tgctgtgtgc ctgcaccacc |
| | ccactgcctt ccttcagcac ctttagctgc |
| 1201 | atttgtatgc cagtgcttaa cacattgcct |
| | tattcatact agcatgctca tgaccaacac |
| 1261 | atacacgtca tagaagaaaa tagtggtgct |
| | tctttctgat ctctagtgga gatctctttg |
| 1321 | actgctgtag tactaaagtg tacttaatgt |
| | tactaagttt aatgcctggc cattttccat |
| 1381 | ttatatatat tttttaagag gctagagtgc |
| | ttttagcctt ttttaaaaac tccatttata |
| 1441 | ttacatttgt aaccatgata ctttaatcag |
| | aagcttagcc ttgaaattgt gaactcttgg |
| 1501 | aaatgttatt agtgaagttc gcaactaaac |
| | taaacctgta aaattatgat gattgtattc |
| | ccccctgaaa aaaaaaaaaa aaaaaaaaaa |
| 1621 | aaaa |

Coding sequence (963 bp) is highlighted.

Codon optimized sequence (963 bp) for full length human annexin A5 protein expression in E. coli bacteria was made and synthesized by Blue Heron Biotech (Bothell, WA, USA).

ATGGCGCAAGTTTTACGTGGGACGGTTACCGATTTTCCGG
GCTTTGATGAACGCGCGGATGCTGAGACCTTACGCAAAGC
GATGAAGGGCCTGGGTACTGACGAGGAGTCCATTCTGACC
CTCCTGACCTCGCGCTCTAACGCTCAACGCCAAGAAATTA
GTGCAGCCTTTAAGACTCTGTTCGGACGTGATTTGCTGGA
CGATCTTAAATCAGAATTAACTGGCAAGTTTGAAAAACTG
ATTGTTGCACTCATGAAACCGTCGCGCCTGTATGATGCAT
ATGAACTGAAACACGCTCTGAAGGGCGCGGGTACTAATGA
GAAAGTGCTGACTGAAATCATTGCGTCCCGTACCCCAGAA
GAACTGCGCGCCATTAAGCAAGTTTATGAGGAGGAGTACG
GTTCTAGCCTGGAAGATGATGTCGTAGGGGATACAAGTGG
TTATTATCAACGTATGCTGGTTGTGCTCCTGCAGGCGAAC
CGCGACCCAGATGCGGGCATTGACGAAGCCCAAGTCGAAC
AAGATGCGCAGGCTCTGTTCCAAGCAGGCGAGCTGAAATG
GGGTACCGACGAAGAAAAATTCATTACGATCTTTGGCACA
CGTAGCGTTAGCCACTTACGGAAAGTTTTCGATAAGTACA
TGACGATCAGCGGATTTCAGATTGAAGAAACCATCGACCG
TGAGACGTCTGGTAATCTGGAGCAGTTACTGCTGGCAGTT
GTTAAAAGCATTCGCTCCATTCCGGCGTATTTGGCTGAGA
CGCTGTATTATGCAATGAAGGGTGCCGGCACCGATGATCA
CACCCTTATTCGCGTGATGGTGTCTCGTTCGGAAATTGAT
CTGTTTAACATTCGGAAAGAGTTTCGTAAGAATTTCGCGA
CGTCATTATATTCGATGATTAAAGGTGACACTTCCGGCGA
TTACAAGAAAGCGTTGCTGTTATTGTGCGGTGAAGATGAT
TAA

The codon optimized sequence was inserted into an OriGene (www.origene.com) bacterial expression vector with N-terminal His tag (pEX-N-His, Cat. #PS100030). This plasmid was transfected into E. coli bacteria to produce recombinant human annexin A5 protein. The N-terminal His tag is cleavable using TEV.

The full-length recombinant human annexin A5 sequence is as follows:

MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD

SARS-CoV-2 spike protein receptor binding domain (RBD) to ACE2 binding inhibitor screening assay was conducted according to the manufacturer's instructions (BD Bioscience, San Diego, CA, USA): Briefly, SARS-CoV-2 spike RBD was coated on a 96-well plate. Next, ACE2-His together with annexin A5 (0-10 μg/ml) were incubated with SARS-CoV-2 spike RBD on the plate. Finally, the plate was treated with Anti-His-HRP followed by addition of an HRP substrate to produce chemiluminescence, which then was measured using a chemiluminescence reader.

Viral infection cellular assay: Human 293T-ACE2 cells were infected with SARS-CoV-2 (Wuhan strain) at an MOI of 0.01 for 2 hours in the presence or absence of varying concentrations of Annexin 5. This MOI induces near complete cytopathogenicity after 72 hours in culture. After 2 hours of infection, virus was removed and cells were incubated with fresh medium containing Annexin 5 for 72 hours. Brightfield images of cells were obtained after 72 hours of infection using an EVOS M7000 imaging system (Thermo Fisher Scientific). Percent survival was measured using the CellTiter-Glo Luminescent Cell Viability Assay (Promega).

### Results

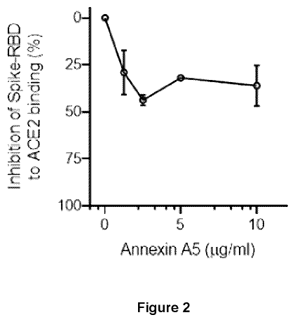

Effects of recombinant human annexin A5 on spike protein receptor binding domain (RBD) to angiotensin converting enzyme 2 (ACE2) binding was examined. FIG. 2 shows that annexin A5 inhibits binding of spike RBD to ACE2 at doses as low as about 0.02 μg/ml, with 1 μg/ml being the most effective tested dose.

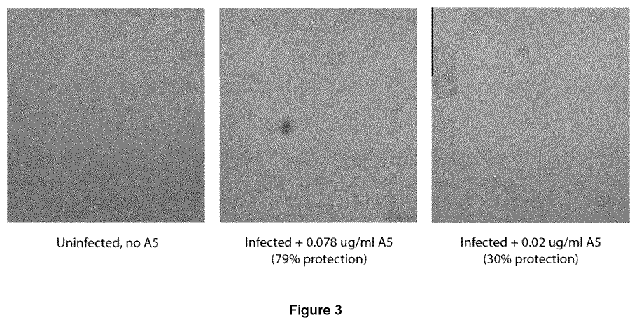

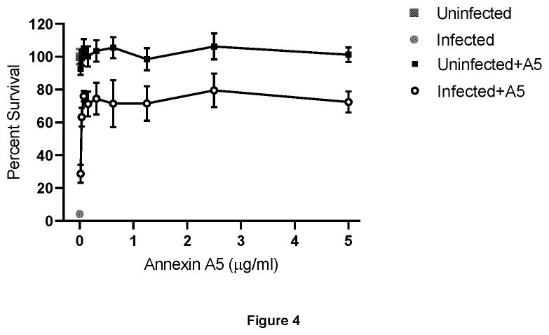

Effects of recombinant human annexin A5 on survival of human 293T-ACE2 cells infected with SARS-CoV-2 were examined. FIGS. 3 and 4 show that untreated and uninfected cells were healthy. Infected cells that were not treated with annexin A5 had very low survival (not shown in FIG. 2). Cells treated with annexin A5 doses of about 0.02 μg/ml or more exhibited 85% or more survival.

These data show that recombinant human annexin A5 inhibits spike-RBD binding to ACE2 and protects against cell death caused by SARS-CoV-2 infection. Our results suggest that annexin A5 may have therapeutic potential in preventing SARS-CoV-2 infection and as a treatment of COVID-19.

### Example 3: Effects of Recombinant Human Annexin A5 on the Spike Protein:ACE2 Interaction

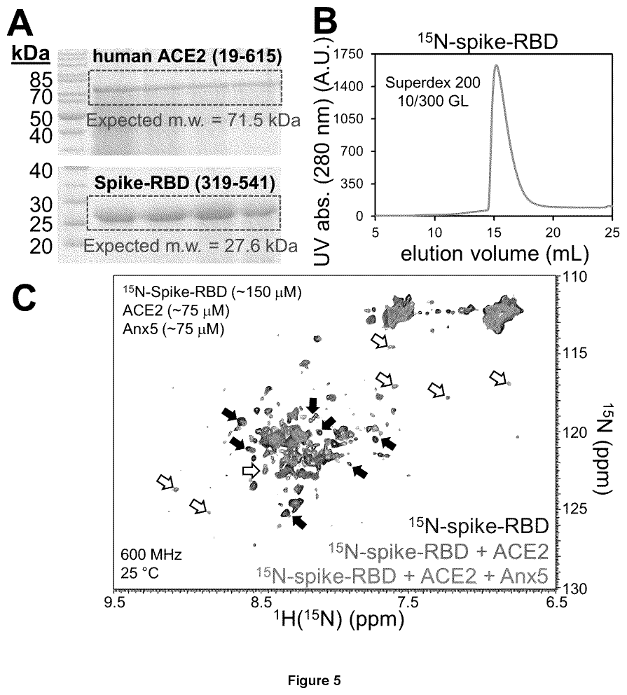

High levels of uniformly ¹⁵N-labeled spike-RBD and human ACE2 extracellular domain were successfully expressed (FIG. 5A, 5B). A direct interaction between these proteins by solution NMR-monitored amide (¹H-¹⁵N) chemical shift changes (FIG. 5C) was demonstrated. Notably, addition of recombinant human annexin A5 into the binary mixture caused several ¹H-¹⁵N peaks unique to the complex to change position and intensity, consistent with a profound effect of annexin A5 on the spike-RBD:ACE2 interaction.

### Example 4: Recombinant Human Annexin A5 Inhibits the SARS-CoV-2 Nucleocapsid (N) Gene Expression in 293Tcells

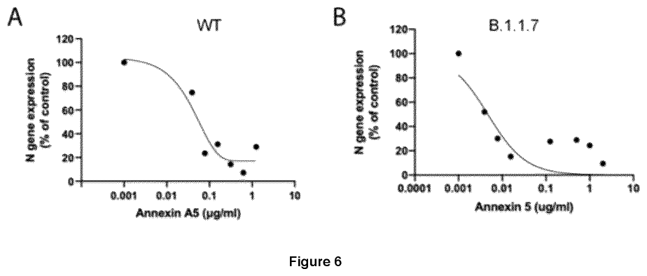

Human 293T-ACE2 cells were infected with SARS-CoV-2 wildtype (WT, original Wuhan strain) or B.1.1.7 variant strain in the presence or absence of annexin A5 (0-5 μg/ml). To assess viral load, qPCR analysis of SARS-CoV-2 nucleocapsid (N) gene levels was performed at 48 hours after viral infection. FIG. 6 shows that recombinant human annexin A5 inhibited the N gene expression in 293T-ACE2 cells infected by either WT (A) or the B.1.1.7 variant (B) over 90%, suggesting that annexin A5 reduces viral load in both WT and B.1.1.7 variant infections in human 293T-ACE2 cells.

The above disclosure generally describes the present invention. Although specific terms have been employed herein, such terms are intended in a descriptive sense and not for purposes of limitation.

All publications, patents and patent applications cited above are herein incorporated by reference in their entirety to the same extent as if each individual publication, patent or patent application was specifically and individually indicated to be incorporated by reference in its entirety.

Although preferred embodiments of the invention have been described herein in detail, it will be understood by those skilled in the art that variations may be made thereto without departing from the spirit of the invention or the scope of the appended claims.

What is claimed is:

1. Annexin A5 for interacting with angiotensin converting enzyme 2 (ACE2).

2. The annexin A5 of claim 1, wherein the annexin A5 binds to ACE2.

3. The annexin A5 of claim 2, wherein annexin A5 binding to ACE2 does not substantially affect ACE2 enzyme activity.

4. The annexin A5 of claim 1, wherein the annexin A5 inhibits interaction of another molecule with ACE2, such as a virus or viral protein.

5. The annexin A5 of claim 4, wherein the annexin A5 inhibits interaction of a coronavirus or coronavirus protein with ACE2.

6. The annexin A5 of claim 5, wherein the annexin A5 inhibits interaction of a coronavirus spike protein with ACE2.

7. Annexin A5 as a SARS-CoV-2 research tool.

8. Annexin A5 as an ACE2 research tool.

9. The annexin A5 of any one of claims 1 to 8, wherein the annexin A5 is human.

10. The annexin A5 of any one of claims 1 to 9, wherein the annexin A5 is recombinant.

11. The annexin A5 of any one claims 1 to 10, wherein the annexin A5 comprises an amino acid sequence having at least 80% sequence identity to SEQ ID NO:1:
MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEITASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD
or an active fragment thereof.

12. The annexin A5 of claim 11, wherein the annexin A5 comprises the amino acid sequence of SEQ ID NO:1.

13. The annexin A5 of claim 12, wherein the annexin A5 consists of the amino acid sequence of SEQ ID NO:1.

14. The annexin A5 of any one of claims 1 to 13, wherein the annexin A5 is bound to a moiety, such as a therapeutic agent, detectable agent, and/or targeting agent, optionally wherein the moiety is a peptide or small molecule.

15. A composition comprising the annexin A5 of any one claims 1 to 14 and a pharmaceutically acceptable carrier.

16. The composition of claim 15, wherein the composition is an inhalable composition or a nasal spray.

17. A method for preventing and/or treating SARS-CoV-2, the method comprising administering annexin A5 to a subject in need thereof.

18. The method of claim 17, wherein the annexin A5 is human.

19. The method of claim 17 or 18, wherein the annexin A5 is recombinant.

20. The method of any one claims 17 to 19, wherein the annexin A5 comprises an amino acid sequence having at least 80% sequence identity to SEQ ID NO:1:
MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEIIASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD
or an active fragment thereof.

21. The method of claim 20, wherein the annexin A5 comprises the amino acid sequence of SEQ ID NO:1.

22. The method of claim 21, wherein the annexin A5 consists of the amino acid sequence of SEQ ID NO:1.

23. The method of any one of claims 17 to 22, wherein the annexin A5 is bound to a moiety, such as a therapeutic agent, detectable agent, and/or targeting agent, optionally wherein the moiety is a peptide or small molecule.

24. The method of any one of claims 17 to 23, wherein the annexin A5 is inhaled or a nasal spray.

25. The method of any one of claims 17 to 24, wherein the subject has a chronic condition such as hypertension or diabetes, is over age 60, and/or is taking an ACE inhibitor.

26. The method of any one of claims 17 to 25, wherein the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.1 to about 1 μg/ml, once, twice, three, or 4 times or more daily or once, twice, three, four, five, or six times weekly, or upon appearance of symptoms, or upon a suspected or known exposure.

27. A method for preventing and/or treating a coronavirus infection and/or for reducing viral load in a coronavirus infection, the method comprising administering annexin A5 to a subject in need thereof.

28. The method of claim 27, wherein the coronavirus infection comprises HCoV-229E, HCoV-0043, HCoV-NL63, HCoV-HKU1, SARS-CoV, MERS-CoV, or SARS-CoV-2.

29. The method of claim 27 or 28, wherein the annexin A5 is human.

30. The method of any one of claims 27 to 29, wherein the annexin A5 is recombinant.

31. The method of any one claims 27 to 30, wherein the annexin A5 comprises an amino acid sequence having at least 80% sequence identity to SEQ ID NO:1:
MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEITASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD
or an active fragment thereof.

32. The method of claim 31, wherein the annexin A5 comprises the amino acid sequence of SEQ ID NO:1.

33. The method of claim 32, wherein the annexin A5 consists of the amino acid sequence of SEQ ID NO:1.

34. The method of any one of claims 27 to 33, wherein the annexin A5 is bound to a moiety, such as a therapeutic agent, detectable agent, and/or targeting agent, optionally wherein the moiety is a peptide or small molecule.

35. The method of any one of claims 27 to 34, wherein the annexin A5 is inhaled or a nasal spray.

36. The method of any one of claims 27 to 35, wherein the subject has a chronic condition such as hypertension or diabetes, is over age 60, and/or is taking an ACE inhibitor.

37. The method of any one of claims 27 to 36, wherein the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.1 to about 1 μg/ml, once, twice, three, or 4 times or more daily or once, twice, three, four, five, or six times weekly, or upon appearance of symptoms, or upon a suspected or known exposure.

38. A method for preventing and/or treating a symptom of a coronavirus infection, the method comprising administering annexin A5 to a subject in need thereof.

39. The method of claim 38, wherein the coronavirus infection comprises HCoV-229E, HCoV-0043, HCoV-NL63, HCoV-HKU1, SARS-CoV, MERS-CoV, or SARS-CoV-2.

40. The method of claim 38 or 39, wherein the annexin A5 is human.

41. The method of any one of claims 38 to 40, wherein the annexin A5 is recombinant.

42. The method of any one claims 38 to 41, wherein the annexin A5 comprises an amino acid sequence having at least 80% sequence identity to SEQ ID NO:1:
MAQVLRGTVTDFPGFDERADAETLRKAMKGLGTDEESILT
LLTSRSNAQRQEISAAFKTLFGRDLLDDLKSELTGKFEKL
IVALMKPSRLYDAYELKHALKGAGTNEKVLTEITASRTPE
ELRAIKQVYEEEYGSSLEDDVVGDTSGYYQRMLVVLLQAN
RDPDAGIDEAQVEQDAQALFQAGELKWGTDEEKFITIFGT
RSVSHLRKVFDKYMTISGFQIEETIDRETSGNLEQLLLAV
VKSIRSIPAYLAETLYYAMKGAGTDDHTLIRVMVSRSEID
LFNIRKEFRKNFATSLYSMIKGDTSGDYKKALLLLCGEDD
or an active fragment thereof.

43. The method of claim 42, wherein the annexin A5 comprises the amino acid sequence of SEQ ID NO:1.

44. The method of claim 43, wherein the annexin A5 consists of the amino acid sequence of SEQ ID NO:1.

45. The method of any one of claims 38 to 44, wherein the annexin A5 is bound to a moiety, such as a therapeutic agent, detectable agent, and/or targeting agent, optionally wherein the moiety is a peptide or small molecule.

46. The method of any one of claims 38 to 45, wherein the annexin A5 is inhaled or a nasal spray.

47. The method of any one of claims 38 to 46, wherein the symptom comprises sepsis, septic shock, acute respiratory distress syndrome (ARDS), and/or organ failure, such as heart and/or lung failure.

48. The method of any one of claims 38 to 47, wherein the subject has a chronic condition such as hypertension or diabetes, is over age 60, and/or is taking an ACE inhibitor.

49. The method of any one of claims 38 to 48, wherein the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.1 to about 1 μg/ml, once, twice, three, or 4 times or more daily or once, twice, three, four, five, or six times weekly, or upon appearance of symptoms, or upon a suspected or known exposure.

50. A method for preventing and/or treating an ACE2-mediated infection, the method comprising administering annexin A5 to a subject in need thereof.

51. The method of claim 50, wherein the ACE2-mediated infection is a coronavirus.

52. The method of claim 51, wherein the coronavirus is HCoV-229E, HCoV-0043, HCoV-NL63, HCoV-HKU1, SARS-CoV, MERS-CoV, or SARS-CoV-2.

53. The method of any one of claims 50 to 52, wherein the annexin A5 is human.

54. The method of any one of claims 50 to 53, wherein the annexin A5 is recombinant.

55. The method of any one claims 50 to 54, wherein the annexin A5 comprises an amino acid sequence having at least 80% sequence identity to SEQ ID NO:1, or an active fragment thereof.

56. The method of claim 55, wherein the annexin A5 comprises the amino acid sequence of SEQ ID NO:1.

57. The method of claim 56, wherein the annexin A5 consists of the amino acid sequence of SEQ ID NO:1.

58. The method of any one of claims 50 to 57, wherein the annexin A5 is bound to a moiety, such as a therapeutic agent, detectable agent, and/or targeting agent, optionally wherein the moiety is a peptide or small molecule.

59. The method of any one of claims 50 to 58, wherein the annexin A5 is inhaled or a nasal spray.

60. The method of any one of claims 50 to 59, wherein the subject has a chronic condition such as hypertension or diabetes, is over age 60, and/or is taking an ACE inhibitor.

61. The method of any one of claims 50 to 60, wherein the annexin A5 is used at a dose of from about 0.001 to about 10 μg/ml, such as from about 0.001, 0.005, 0.01, 0.02, 0.05, 0.1, 0.5, 1, 1.5, 2, 3, 4, 5, 6, 7, 8, or 9 to about 0.005, 0.01, 0.02, 0.05, 0.1, 0.5, 1, 1.5, 2, 3, 4, 5, 6, 7, 8, 9, or 10 μg/ml, such as from about 0.1 to about 1 μg/ml, once, twice, three, or 4 times or more daily or once, twice, three, four, five, or six times weekly, or upon appearance of symptoms, or upon a suspected or known exposure.
